(19) United States
(12) Patent Application Publication
(10) Pub. No.: US 2009/0287083 A1
(43) Pub. Date: Nov. 19, 2009

(54) Cavitation detector

(76) Inventors: Leonid Kushculey, Rehovot (IL); Vladimir Goland, Ashdod (IL)

Correspondence Address: EITAN MEHULAL LAW GROUP, 10 Abba Eban Blvd. PO Box 2081, Herzlia, 46120, IL

(21) Appl. No.: 12/153,085

(22) Filed: May 14, 2008

(51) Int. Cl. A61B 8/00

(52) U.S. Cl. 600/449

(57) ABSTRACT

A method for detecting cavitation in an adipose tissue, the method comprising computing a level of correlation between at least two received ultrasonic signals, wherein the level of correlation is indicative of cavitation.

### FIELD OF THE DISCLOSURE

Embodiments of the disclosure relate to a cavitation detector usable in a non-invasive body contouring procedure.

### BACKGROUND

A non-invasive body contouring procedure is often defined as the destruction of subcutaneous adipose tissues using focused ultrasound energy, such as HIFU (High-Intensity Focused Ultrasound). This procedure sometimes serves as an alternative to traditional liposuction procedures.

Commonly, focused ultrasound energy may destroy adipose tissues using two major mechanisms—thermal and mechanical. In the thermal mechanism, absorption of ultrasonic energy in the treated tissue causes its heating and, eventually, destruction. In the mechanical mechanism, various forces created by the ultrasonic energy may cause cavitation, fractionation, shearing, tension and/or liquefaction of the adipose tissue, which lead to its destruction. The mechanical mechanism of adipose tissue destruction is commonly referred to as “histotripsy”.

Cavitation, one of the phenomena belonging to the mechanical destruction mechanism, is commonly defined as the formation of micro-bubbles within the treated tissue, which cause its disintegration. The cavitation process is usually very dynamic, and may be characterized by rapid creation and collapse of the micro-bubbles.

### SUMMARY OF THE DISCLOSURE

There is provided, in accordance with an embodiment of the disclosure, a method for detecting cavitation in an adipose tissue, the method comprising computing a level of correlation between at least two received ultrasonic signals, wherein the level of correlation is indicative of cavitation.

In some embodiments, a level of correlation lower than a predetermined threshold indicates a cavitation event.

In some embodiments, the method further comprises calculating a ratio between a number of cavitation events and a number of radiated ultrasonic signals, to determine a ratio of cavitation.

In some embodiments, the method further comprises computing an intensity of the cavitation event.

In some embodiments, the method further comprises computing a location of the cavitation event.

In some embodiments, the location of the cavitation pertains to a one-dimensional location.

In some embodiments, the location of the cavitation pertains to a two-dimensional location.

In some embodiments, the location of the cavitation pertains to a three-dimensional location.

There is provided, in accordance with an embodiment of the disclosure, a method for automatically controlling a histotripsy appliance, the method comprising adjusting a histotripsy procedure parameter based on an indication of cavitation.

In some embodiments, the histotripsy procedure parameter is an electrical power applied to the histotripsy appliance for radiating focused ultrasonic signals.

In some embodiments, the histotripsy procedure parameter is a voltage applied to the histotripsy appliance for radiating focused ultrasonic signals.

In some embodiments, the histotripsy procedure parameter is a focus of the histotripsy appliance.

In some embodiments, the histotripsy procedure parameter is a number of focused ultrasonic signals radiated by the histotripsy appliance.

There is provided, in accordance with an embodiment of the disclosure, an ultrasonic apparatus for lysing an adipose tissue, the apparatus comprising a transducer adapted to radiate focused ultrasonic signals; and a controller adapted to compute a level of correlation between at least two received ultrasonic signals, wherein the level of correlation is indicative of cavitation.

In some embodiments, a level of correlation lower than a predetermined threshold indicates a cavitation event.

In some embodiments, said controller is further adapted to compute a ratio between a number of cavitation events and a number of radiated ultrasonic pulses, to determine a ratio of cavitation.

In some embodiments, said controller is further adapted to compute an intensity of the cavitation event.

In some embodiments, said controller is further adapted to compute a location of the cavitation event.

In some embodiments, the location of the cavitation pertains to a one-dimensional location.

In some embodiments, the location of the cavitation pertains to a two-dimensional location.

In some embodiments, the location of the cavitation pertains to a three-dimensional location.

In some embodiments, said transducer is further adapted to function as a receiver for receiving said received ultrasonic signals.

In some embodiments, the ultrasonic apparatus further comprises a cavitation detector adapted to receive said received ultrasonic signals.

In some embodiments, the ultrasonic apparatus further comprises a cavitation detector adapted to receive said received ultrasonic signals

In some embodiments, said transducer is a multi-element transducer.

In some embodiments, at least one element of said multi-element transducer is adapted to receive the ultrasonic reflection signals.

In some embodiments, said transducer is associated with a time-reversal-based histotripsy system.

In some embodiments, said controller is further adapted to adjust a histotripsy procedure parameter.

In some embodiments, said histotripsy procedure parameter is an electrical power used for radiating the focused ultrasonic signals.

In some embodiments, said histotripsy procedure parameter is a voltage used for radiating the focused ultrasonic signals.

In some embodiments, said histotripsy procedure parameter is a focus of said transducer.

In some embodiments, said histotripsy procedure parameter is a number of the focused ultrasonic signals radiated by said transducer.

In some embodiments, said controller is further adapted to notify a user of a cavitation parameter.

In some embodiments, said cavitation parameter is a cavitation event.

In some embodiments, said cavitation parameter is an intensity of a cavitation event.

In some embodiments, said cavitation parameter is a ratio between a number of cavitation events and a number of radiated ultrasonic signals.

In some embodiments, said cavitation parameter is a location of a cavitation event.

In some embodiments, the ultrasonic apparatus further comprises a monitor adapted to display the notification of the cavitation parameter.

In some embodiments, the ultrasonic apparatus further comprises a speaker adapted to sound the notification of the cavitation parameter.

In some embodiments, the ultrasonic apparatus further comprises a vibrator adapted to sensorially relay the notification of the cavitation parameter to the user.

### BRIEF DESCRIPTION OF THE FIGURES

Examples illustrative of embodiments of the disclosure are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1A schematically shows a transducer;

FIG. 1B schematically shows a transducer and cavitation detectors;

FIG. 1C schematically shows a transducer and a cavitation detector;

FIG. 2A schematically shows a transducer, a resonator and cavitation detectors;

FIG. 2B schematically shows transducers, a resonator and cavitation detectors;

FIG. 3 schematically shows a multi-element transducer;

FIG. 4 schematically shows a flow chart of a cavitation detection process;

FIG. 5 schematically shows an ultrasonic pulse;

FIG. 6 schematically shows reflected ultrasonic signals;

FIG. 7 schematically shows correlation coefficient graphs;

FIG. 8 schematically shows correlation coefficient graphs;

FIG. 9 schematically shows reflected ultrasonic signals;

FIG. 10 schematically shows correlation coefficient graphs;

FIG. 11 schematically shows correlation coefficient graphs;

FIG. 12 schematically shows one-bit versions of correlation coefficient graphs;

FIG. 13 schematically shows one-bit versions of correlation coefficient graphs;

FIG. 14A schematically shows a cavitation vector;

FIG. 14B schematically shows a ratio of cavitation vector;

FIG. 15 schematically shows an exemplary treatment of a patient by a caregiver; and

FIG. 16 schematically shows a block diagram of an ultrasonic apparatus.

### DETAILED DESCRIPTION OF EMBODIMENTS

An aspect of some embodiments of the disclosure relates to a cavitation detector adapted to detect cavitation within an adipose tissue. The detected cavitation may be the result of a histotripsy procedure wherein focused ultrasound energy is emitted from a transducer towards an adipose tissue in order to cause its destruction.

The following detailed description discloses a method for analyzing ultrasonic signals reflected from a treated adipose tissue and/or from its surroundings. A correlation algorithm applied to the reflected ultrasonic signals may detect cavitation within the tissue. In addition, methods, devices and systems that utilize results of the algorithm and/or of the signal analysis are also disclosed. For example, the detailed description discloses embodiments for visually and/or sonically relaying cavitation-related information to a user. Additional examples are usage of the results for estimating a location of the cavitation, for computing a ratio of cavitation (no. of cavitation events per no. of pulses) and/or for automatically controlling treatment parameters, such as electrical power, number of pulses and focus.

The detailed description further discloses configurations of a cavitation detector (or, in some embodiments, multiple detectors) adapted to operate in conjunction with an ultrasonic transducer (or, in some embodiments, multiple transducer elements). In some embodiments, an ultrasonic transducer also serves as the detector.

For clarity of presentation, the detailed description begins with a description of the cavitation detector(s) configurations and proceeds with the method for analyzing reflected ultrasonic signals and the related methods, devices and systems.

### Cavitation Detector Configurations

During a histotripsy procedure, focused ultrasonic signals are often radiated from a piezoelectric transducer towards an adipose tissue whose destruction is desired.

According to some embodiments, a cavitation detector, functioning as a receiver, receives ultrasonic signals which are essentially the signals previously radiated and now being reflected towards the receiver. The received ultrasonic signals may then be processed and/or analyzed for estimating cavitation and/or for performing related tasks.

As referred to herein, the term “reflection” may refer to an ultrasonic signal which is essentially a signal irradiated from a transducer and now being reflected, either in a focused form or in a scattered form. The reflection of the signal is essentially due to its interaction with the adipose tissue, its surrounding tissue and/or micro-bubbles associated with cavitation.

A resonant frequency of a piezoelectric transducer is often dictated by one or more parameters such as its measurements and/or mass. Thus, a specific transducer may be adapted to receive ultrasonic signals substantially at a same frequency at which it is adapted to radiate ultrasonic signals, making it essentially a “narrowband” transducer. On the contrary, other piezoelectric elements, adapted to function as sensors, receivers and/or microphones, may be formed so as to be essentially “wideband”—capable of receiving ultrasonic signals at a wide range of frequencies.

Therefore, a histotripsy transducer also used as a receiver for the cavitation detection capability, may be indifferent to reflected ultrasonic signals which are substantially beyond its resonant frequency. Wideband receivers, on the other hand, may be able to sense such reflected ultrasonic signals at a broader range of frequencies. It should be noted, however, that a histotripsy transducer may be formed such that it is able to sense reflected ultrasonic signals at a number of frequencies or at a range of frequencies. For example, a transducer may be formed having different thicknesses in different regions of its surface, so that each of these regions may be adapted to sense (and essentially to radiate) ultrasonic signals at a different frequency. As another example, a transducer may be formed as a multi-element transducer, wherein each element may function as a separate transducer and some elements may have different thicknesses enabling them to sense different frequencies.

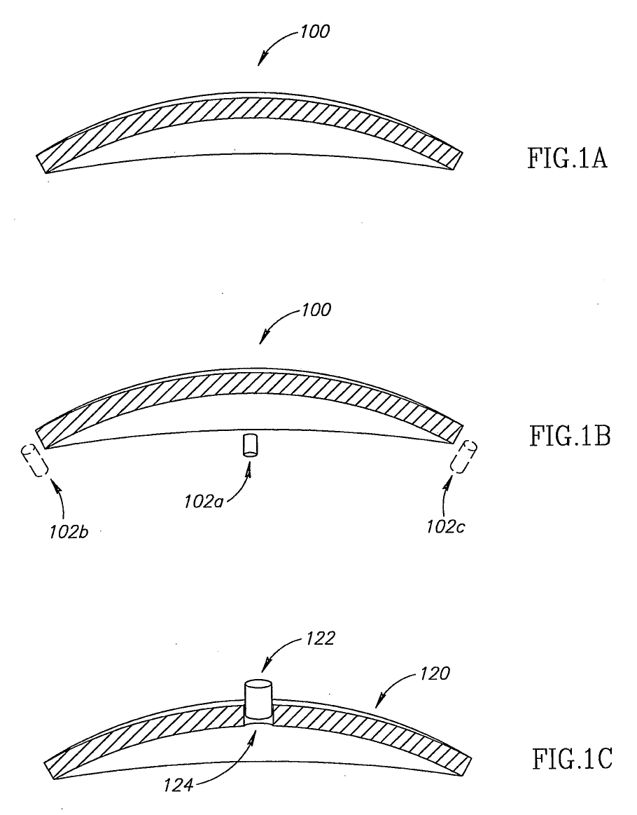

In an embodiment, a transducer usable for radiating focused ultrasonic signals serves also as a cavitation detector. Reference is now made to FIG. 1A, which shows a cross-sectional view of a transducer 100. Transducer 100 is shown dome-shaped, although persons of skill in the art will recognize the applicability of this disclosure to otherwise-shaped transducers. Transducer 100 may be adapted to both radiate and receive ultrasonic signals, the radiating essentially aimed at destroying adipose tissue while the receiving essentially aimed at detecting cavitation. For example, transducer 100 may radiate one or more of focused ultrasonic signals towards an adipose tissue, and then receive one or more reflections and/or scattering of these signal(s), referred to as “received ultrasonic signal(s)”. The received reflections may then be processed and/or analyzed.

In an embodiment, one or more cavitation detectors may be functionally coupled to a transducer. Reference is now made to FIG. 1B, which shows a cross-sectional view of transducer 100 of FIG. 1A, functionally coupled to a first cavitation detector 102a (hereinafter “first detector”). Optionally, additional one or more cavitation detectors, such as a second detector 102b and/or a third detector 102c are functionally coupled to transducer 100. Furthermore, persons of skill in the art will recognize that more than three cavitation detectors may be functionally coupled to transducer 100.

Transducer 100 may be adapted to radiate focused ultrasonic signals, and optionally to receive ultrasonic signals. Detectors 102a-c may be essentially passive—adapted to receive ultrasonic signals, or active—adapted to radiate and/or to receive ultrasonic signals.

In an embodiment, a cavitation detector is fitted within an aperture in a transducer. Reference is now made to FIG. 1C, which shows a cross-sectional view of a transducer 120. Transducer 120, similar to transducer 100 of FIG. 1A, is shown dome-shaped, although persons of skill in the art will recognize the applicability of this disclosure to otherwise-shaped transducers. An aperture 124 may be formed in transducer 120, optionally in its center, and a detector 122 may be fitted within the aperture. In addition to detector 122, additional one or more detectors (not shown) may be functionally coupled to transducer 120.

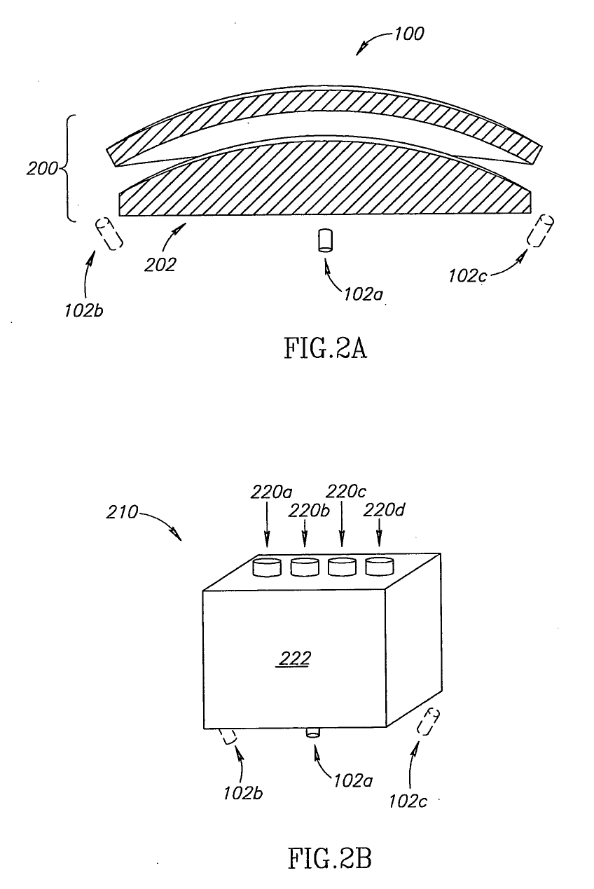

In an embodiment, one or more cavitation detectors are functionally coupled to a time-reversal-based histotripsy system which includes a transducer and a resonator. In time-reversal-based histotripsy systems, wherein a special signal processing method is employed for focusing radiated ultrasonic signals, the resonator is often placed beneath the transducer. Reference is now made to FIG. 2A, which shows a histotripsy system 200 including transducer 100 of FIG. 1A and a resonator 202. Resonator 202 may be a dome-shaped device, essentially matching the dome shape of transducer 100. One or more cavitation detectors, such as detectors 102a-b of FIG. 1A, may be positioned substantially beneath resonator 202, so that they can sense reflected ultrasonic signals before these signals are distorted by the resonator.

FIG. 2B shows another embodiment of a time-reversal-based histotripsy system 210, which includes one or more transducers, such as transducers 220a-d, positioned over a resonator 222. Similar to FIG. 2A, one or more cavitation detectors, such as detectors 102a-b of FIGS. 1A and 2A, may be positioned substantially beneath resonator 222, so that they can sense reflected ultrasonic signals before these signals are distorted by the resonator. Time reversal ultrasound focusing is further explained in applicants' U.S. patent application Ser. No. 12/003,811 which discloses, inter alia, a method for producing a time reversed signal for destroying a fat tissue, the method comprising emitting an electrical signal towards a transducer unit being essentially in contact with a tissue simulating medium; receiving, using a sensor submerged within the tissue simulating medium, an ultrasonic signal derived from at least the electrical signal; converting the ultrasonic signal to a digital signal; and time-reversing the digital signal to produce a time-reversed signal. Optionally, the transducer unit comprises at least one transducer attached to at least one resonator. Optionally, the method further comprises storing the time-reversed signal in a memory, along with a corresponding datum pertaining to a relative location of the transducer unit and the sensor.

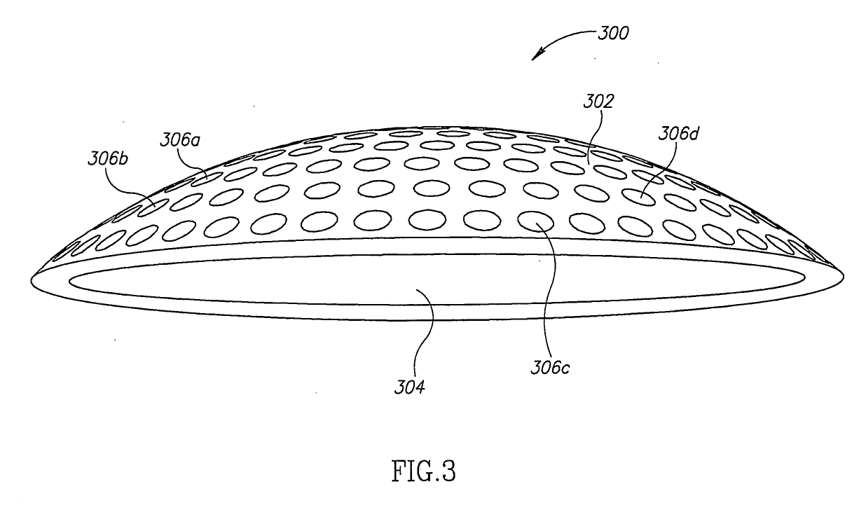

In an embodiment, at least one element of a multi-element transducer serves as a cavitation detector. Reference is now made to FIG. 3, which shows a multi-element transducer 300 having a plurality of elements, represented by electrodes such as electrodes 306a-d. Similar to transducer 100 of FIG. 1A, multi-element transducer 300 is shown dome-shaped, although persons of skill in the art will recognize the applicability of this disclosure to otherwise-shaped multi-element transducers. Electrodes 306a-d and other shown but non-referenced electrodes, despite being shown on a top surface 302 of transducer 300, may also be positioned on a bottom surface 304 of the transducer, or on both surfaces. Some or all of electrodes 306a-d and/or other shown but non-referenced electrodes are shown essentially circular, but may have different shapes than shown. Multi-element transducers are further explained in applicants' U.S. patent application Ser. Nos. 12/081,378 and 12/081,379, entitled, respectively, “Patterned Ultrasonic Transducers” and “Operation of Patterned Ultrasonic Transducers”.

At least one element, represented by an electrode such as any of electrodes 306a-d, may serve as a cavitation detector adapted to receive ultrasonic signals. The at least one cavitation detector may be assigned solely for detecting cavitation and not for irradiating focused ultrasonic signals. Alternatively, the at least one cavitation detector may be adapted to both detecting cavitation and irradiating focused ultrasonic signals. Another option is designation of one or more elements solely for cavitation detection, one or more other elements both for cavitation detection and irradiation of signals, and optionally one or more other elements solely for irradiation of signals.

### Method for Analyzing Reflected Ultrasonic Signals

In an embodiment, ultrasonic signals reflected substantially from an adipose tissue are processed and/or analyzed, essentially for detecting cavitation within the tissue.

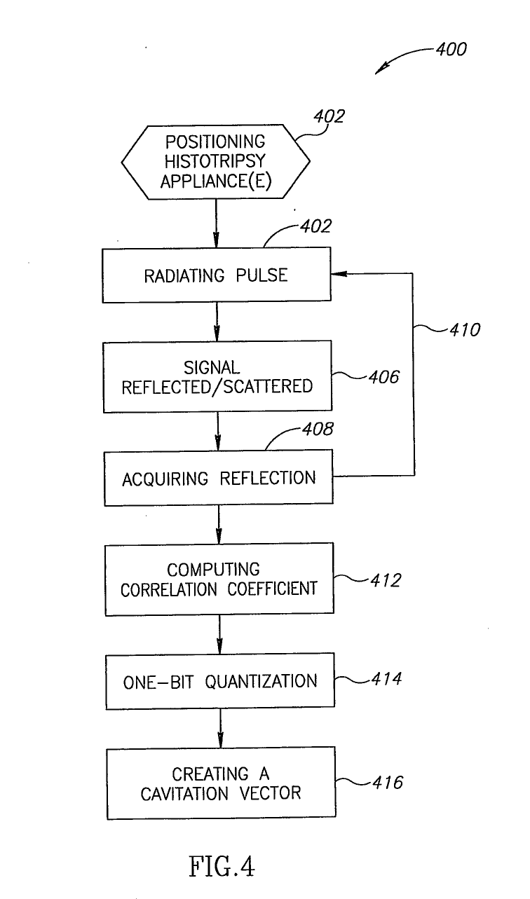

Reference is now made to FIG. 4, which shows a flow chart of a cavitation detection process 400. In a block 402, one or more histotripsy appliances are positioned substantially above a treatment area of a patient. The one or more histotripsy appliances may include one or more transducers, one or more cavitation detectors, and/or one or more transducers adapted to operate as cavitation detectors—examples of all of which are shown in FIGS. 1A-C, 2A-B and 3.

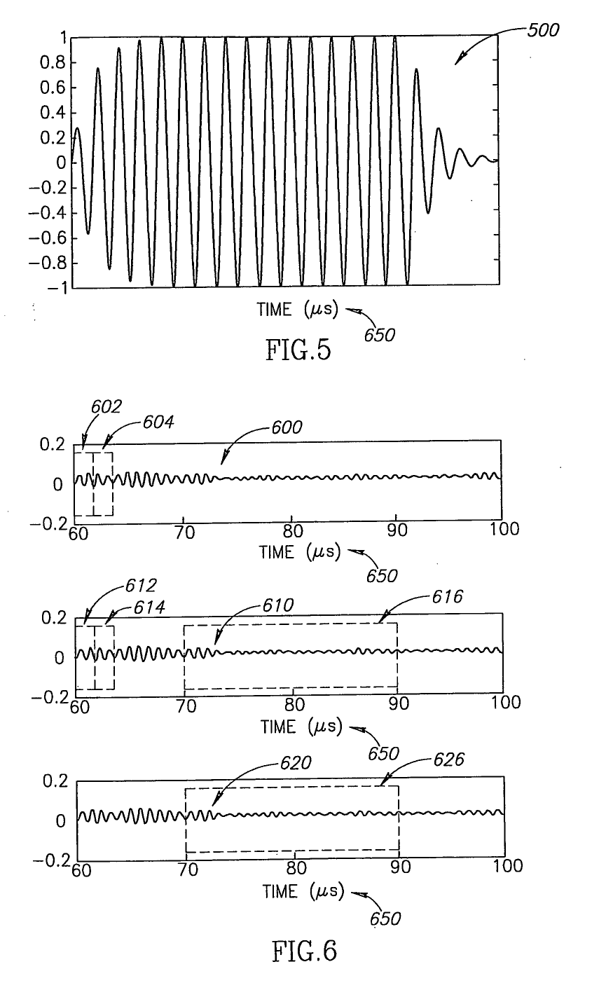

In a block 404, an ultrasonic pulse is radiated from one or more transducers of the histotripsy appliance(s). Reference is now made to FIG. 5, which shows an exemplary radiated ultrasonic pulse 500. The exemplary ultrasonic pulse 500 is shown approximately 20 μs-long, having 20 central wave periods at 1 MHz, but may have a different length, a different number of periods, a different frequency and/or the like.

In a block 406, the ultrasonic pulse may hit tissues substantially in the treatment area. The pulse, constituting what is referred to as an ultrasonic signal, may then scatter, and a portion of which may be reflected towards the one or more transducers and/or one or more cavitation detectors.

In a block 408, at least one of the one or more cavitation detectors receives (hereinafter “acquires”) an ultrasonic signal reflected towards it (hereinafter a “reflected ultrasonic signal”, a “reflection” or a “received ultrasonic signal”). The acquired reflected ultrasonic signal may be referred to as a unitary “acquisition”—a reflected ultrasonic signal received over a pre-defined period of time (hereinafter “length”). The acquisition may be digitized, to allow for its future processing and/or analysis.

Optionally, a length of an acquisition is set such that it contains a meaningful signal, reflected substantially from an estimated cavitation zone, so that conclusions as to existence or non-existence of cavitation in that zone may be drawn. When using a dome-shaped transducer, for example, a meaningful signal is likely to be reflected from an area around a focal point of the transducer—since a majority of the energy radiated by the transducer is aimed at that point.

In an exemplary experiment performed by the inventors, a dome-shaped 1 MHz transducer having a focal length of 54 mm was used. The transducer used in the experiment is schematically similar to transducer 120 of FIG. 1C, including aperture 124 and detector 122, and the proceedings of the experiment are therefore described herein with reference to transducer 120.

A received ultrasonic signal is likely to be arriving from the focal point of transducer 120 if it is acquired approximately 2d/v seconds after its original signal is radiated, where d is the focal length (in meters) and v is the medium sound speed (in m/s). In the case of an adipose tissue, v is approximately equal to the sound speed in water, which is about 1500 m/s. In the experiment, where the focal length was 54 mm (or 0.054 meters), a reflection from the focal point was acquired approximately 0.000072 seconds, or 72 μs, after the original signal's radiation. Since the ultrasonic pulse radiated in block 404 is approximately 20 μs-long, the reflection from the focal point during the experiment was acquired over a same period of approximately 20 μs, so that the reflection ended essentially 92 μs after the original signal's radiation. Generally, a reflection of an ultrasonic pulse radiated at time T₀ and which lasts X units of time, will essentially start being received at its source at time T_{0+(2d/v)}, will last the same X units of time, and will stop being received at time T_{0+(2d/v)}+X.

In addition to acquiring signals reflected from the focal point, it may be desired to acquire signals arriving from areas closer to and/or farther away from transducer 120, in order to detect cavitation also in these areas. Therefore, an acquisition should optionally cover an extended period of time, starting earlier than T_{0+(2d/v)} and ending later than T_{0+(2d/v)}+X. In the experiment, the length of the extended period was set to approximately 40 μs (2× units of time), covering a period of between 60 and 100 μs from the radiation of a signal to its complete return. Persons of skill in the art will recognize that other lengths may also be applicable, as long as an acquisition contains a meaningful signal.

Another factor that may be considered when setting an acquisition length is a resonant frequency of a transducer in use. Generally, a relatively higher frequency may yield a smaller focal zone (a three-dimensional area surrounding the focal point) and vice versa. Therefore, a relatively high frequency may call to a relatively shorter acquisition length, and a relatively low frequency may call to a relatively longer acquisition length.

Actions and/or occurrences of blocks 404, 406 and 408 may be repeated 410 at least twice, in order to obtain at least two subsequent acquisitions. The term “subsequent acquisitions”, herein, refers to acquisitions performed either immediately one after the other, or not immediately one after the other—having additional one or more acquisitions in between. A minimal number of two acquisition may be required in order to compare between the two and draw meaningful conclusions as to cavitation, as further explained below.

Reference is now made to FIG. 6, which shows three exemplary acquisitions 600, 610 and 620, acquired sequentially during the experiment; acquisition 600 was acquired first, acquisition 610 second, and acquisition 620 third. Acquisitions 600, 610 and 620 were acquired at the very beginning of the experiment, so it is likely that no cavitation was present in the tissue at the time of acquiring.

Acquisitions 600, 610 and 620 are shown with a time scale 650, but may as well be shown with a corresponding distance scale, indicating a distance from a cavitation detector or the like; since distance and time are directly proportionate given a known speed (which is approximately 1500 m/s, as discussed above), they may be used interchangeably when showing acquisitions, such as acquisitions 600, 610 and 620.

Referring now back to FIG. 4, a correlation coefficient is computed in a block 412, using a correlation algorithm. The correlation coefficient may be aimed at determining a level of correlation and/or similarity between at least two subsequent acquisitions, wherein the level of correlation is indicative of cavitation. Since cavitation often includes rapid changing of a treated adipose tissue due to formation of micro-bubbles within it, a change in ultrasonic signals reflected from the tissue over time may suggest the existence of cavitation. From a graphical point of view, the correlation algorithm mathematically expresses an amount of similarity between at least two graphical waveforms of the respective at least two acquisitions. In FIG. 6, for example, as may be observed even with the naked eye, acquisitions 600 and 610 appear very similar, and so do acquisitions 610 and 620. This similarity may indicate lack of cavitation or a low level of cavitation, as further explained below.

Optionally, the correlation coefficient (k) is a number between “−1” and “1” (meaning, −1≧k≧1), where “−1” substantially indicates lack of correlation (and a complete phase shift) and “1” substantially indicates perfect correlation. When the correlation coefficient is closer to “−1” it is said to indicate a relatively low level of correlation, whereas when it is closer to “1” it is said to indicate a relatively high level of correlation. Persons of skill in the art, however, will recognize that the correlation coefficient may be represented by other numerical and/or textual values, essentially indicating a level of correlation.

Optionally, the correlation coefficient is separately computed for a same time window of each of the acquisitions participating in the computation. The time window may cover a temporal portion of each of the participating acquisitions, so that one or multiple adjoining time windows may essentially entirely cover each acquisition. The number of adjoining time windows that may be needed for covering each acquisition is a function of the time window size. Setting and deciding on a time window size may generally involve a tradeoff between micro- and macro-level conclusions that may be drawn from the correlation coefficient. Whereas a relatively small time window may support micro-level conclusions such as a location of cavitation, a relatively large time window may support macro-level conclusions such as an intensity of cavitation. In some cases, however, micro- and macro-level conclusions may be drawn from both a relatively small time window and a relatively large time window.

Time window sizes may be better understood by observing FIG. 6, which shows exemplary time windows. A time window 602 of acquisition 600 and a corresponding time window 612 of acquisition 610 are shown. Time windows 602 and 612 are 2 μs-long, and at the frequency of 1 MHz used in the experiment they each cover two central wave periods. Time windows 602 and 612 are hereinafter referred to as “small” time windows, although a definition of a small time window may also be different than 2 μs, and may be set to fit a transducer frequency and/or other factor(s).

The correlation algorithm may compute a correlation coefficient of corresponding portions of acquisitions 600 and 610, which are positioned within time windows 602 and 612, respectively. Similarly, the correlation algorithm may compute a correlation coefficient using additional, adjoining time windows, such as a time window 604 and a time window 624, until coverage of essentially the entirety of acquisitions 600 and 610 is achieved. Alternatively, the correlation algorithm computes a correlation coefficient using one or more time windows such as time windows 602-602 and 612-614, without covering the entirety of acquisitions such as acquisitions 600 and 610.

An example of “large” time windows is shown at a time window 616 of acquisition 610 and a corresponding time window 626 of acquisition 620. Time windows 616 and 626 are each 20 μs-long, and cover 20 central wave periods each. The 20 μs length corresponds to the length of radiated ultrasonic pulse 500 of FIG. 5, which is also 20 μs, so that essentially an entire reflection of the radiated ultrasonic pulse is processed at once. It should be noted, however, that a definition of a large time window may also be different than 20 μs, and may be set to fit a different length of a radiated ultrasonic pulse, a different transducer frequency and/or other factor(s).

The aforementioned tradeoff between usage of small or large time windows may be overcome by using both. When employing the correlation algorithm on a set of two or more subsequent acquisitions, parallel usage of both small and large time windows may produce two (or more, if more than two sizes of time windows are used) sets of correlation coefficients, each a product of a differently-sized time window. The set of correlation coefficients associated with a small time window will enable drawing conclusions as to, for example, cavitation location, while the set of correlation coefficients associated with a large time window will enable drawing conclusions as to, for example, cavitation intensity.

The correlation algorithm optionally includes a bias mechanism, adapted to filter out relatively weak signals of the participating acquisitions—weak signals that may be merely noise. The bias may include a value which essentially deflects the computed correlation coefficient—it gives a higher significance to high amplitudes than to low amplitudes. The bias value, which may be changed if desired, essentially governs an amount of filtering of the relatively low amplitudes. In a noise-rich environment, for example, the bias value may be adjusted so as to compensate for this noise and produce a more reliable correlation coefficient.

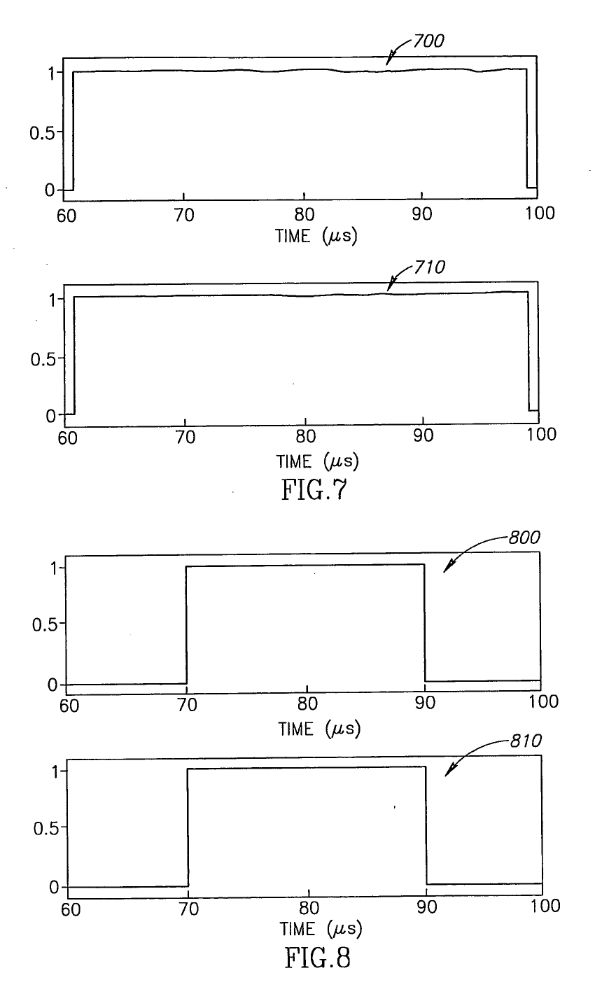

Reference is now made to FIG. 7, which shows a graph 700 of correlation coefficients computed using subsequent acquisitions 600 and 610 of FIG. 6, and a graph 710 of correlation coefficients computed using subsequent acquisitions 610 and 620 of FIG. 6—both graphs computed with the small time window. As shown, in both graphs 700 and 710 the correlation coefficient are essentially “1” or almost “1” throughout substantially the entire period of 60-100 μs.

The correlation value of “1” or almost “1” in both graphs 700 and 710 suggests high level correlation or even a perfect one. This level of correlation in graph 700 may indicate lack of cavitation at the time acquisitions 610 and 620 of FIG. 6 were made. Similarly, it may indicate lack of cavitation at the time acquisitions 620 and 630 of FIG. 6 were made.

Reference is now made to FIG. 8, which shows a graph 800 computed similar to graph 700 of FIG. 7 but with the large time window, and a graph 810 computed similar to graph 700 of FIG. 7—also with the large time window. As shown, computing the correlation algorithm using the large time window yields slightly different results than computation with the small time window. Both graphs 800 and 810 show a correlation coefficient of “1” or almost “1” throughout substantially the entire range of approximately 70 to 90 μs—the range which is included in the large time window. Values of “0” shown prior to 70 μs and after 90 μs do not represent, in this exemplary case, correlation coefficients—since such correlation coefficients were not computed for these periods in the large time window.

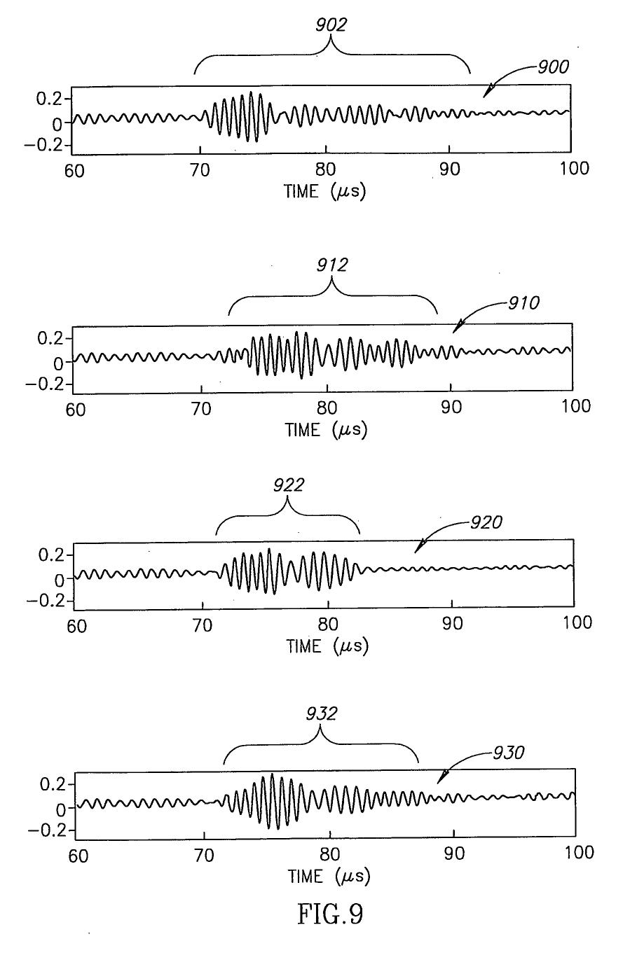

Reference is now made to FIG. 9, which shows four exemplary acquisitions 900-930, acquired sequentially during the experiment; acquisition 900 was acquired first, acquisition 910 second, acquisition 920 third and acquisition 930 fourth. Acquisitions 900-930 demonstrate two phenomena that may be associated with ongoing cavitation. First, as may be observed even with the naked eye, acquisitions 900-930 are graphically different than one another, as opposed to acquisitions 600-620 of FIG. 6—and this variance may indicate the existence of cavitation. Second, acquisitions 900-930 include portions 902-932, respectively, which exhibit amplitudes relatively stronger than amplitudes visible in acquisitions 600-620 of FIG. 6. These stronger amplitudes may indicate the existence of cavitation, since micro-bubbles of the cavitation are often a better reflector than a normal-state adipose tissue.

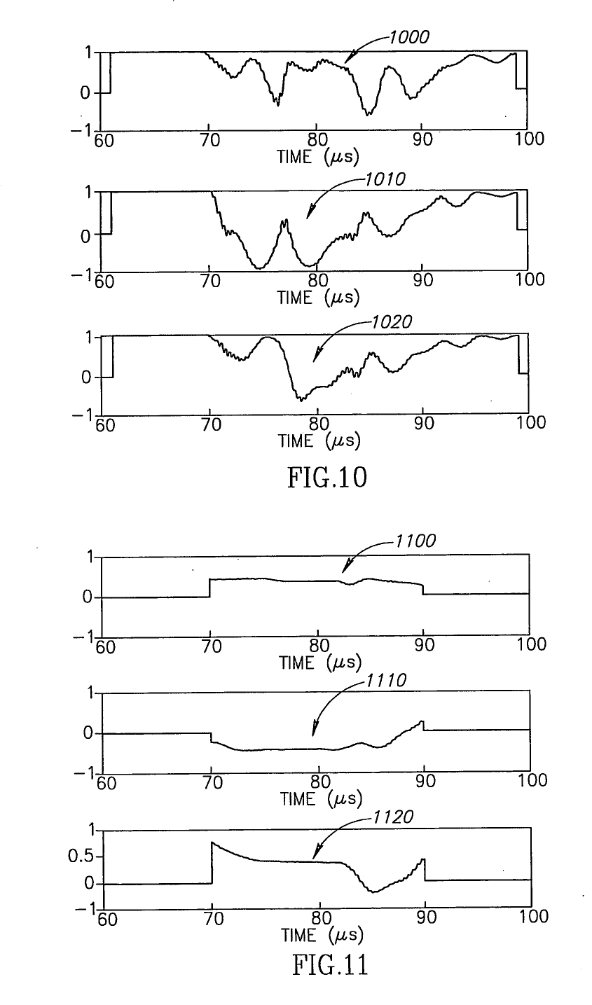

Reference is now made to FIG. 10, which shows three graphs computed with the small time window: A graph 1000 of correlation coefficients computed using subsequent acquisitions 900 and 910 of FIG. 9, a graph 1010 of correlation coefficients computed using subsequent acquisitions 910 and 920 of FIG. 9, and a graph 1020 of correlation coefficients computed using subsequent acquisitions 920 and 930 of FIG. 9.

Graph 1000 shows a correlation coefficient of substantially “1” between approximately 60-70 μs, and various correlation coefficients lower than “1” between approximately 70-100 μs. Therefore, it is likely that cavitation occurred at least in an area located at a location slightly less distant than 54 mm (which corresponds, as mentioned, to 72 μs). This data fits well with the focal length of 54 mm of the transducer used in the experiment.

In graphs 1010 and 1020, similar to graph 1000, correlation coefficients are substantially “1” between approximately 60-70 μs, and are substantially lower than “1” throughout the majority of the remaining duration. This suggests that the location of cavitation remained essentially the same—slightly before 54 mm.

Reference is now made to FIG. 1, which shows three graphs which are similar to graphs 1000-1020 of FIG. 10 but were computed with the large time window: A graph 1100 which is similar to graph 1000, a graph 1110 which is similar to graph 1010, and a graph 1120 which is similar to graph 1020. In graph 1100, correlation coefficients are higher than “0” throughout the entire period of approximately 70-90 μs. In graph 1110, correlation coefficients are lower than “0” throughout almost the entire period of 70-90 μs; at approximately the last 2 μs of this period, the correlation coefficients rise above “0”. In graph 1120, correlation coefficients are higher than “0” throughout the period of 70-85 μs. At approximately 85 μs, the correlation coefficients descend below “0”, and rise back above “0” at approximately 89 μs. Values of “0” shown prior to 70 μs and after 90 μs do not represent, in these exemplary graphs 1010-1020, correlation coefficients—since such correlation coefficients were not computed for these periods in the large time window.

As described, and as may also be observed in graphs 1100-1120 with the naked eye, correlation coefficients of graph 1110 are essentially lower than correlation coefficients of graphs 1100 and 1120. This may indicate relatively low-intensity cavitation in graph 1100, relatively high-intensity cavitation in subsequent graph 1110, and again relatively low-intensity cavitation (which is, in fact, slightly higher in intensity than the cavitation of graph 1100) in graph 1120.

Reference is now made back to FIG. 4. In the description of block 412, computation of correlation coefficients was explained, along with explanations of conclusions than may be drawn from the correlation coefficients as to location and/or intensity of cavitation. In a block 414, the correlation coefficients computed in block 412 are optionally one-bit quantized.

Quantization is often referred to as a restriction of a variable to a specific set of values. In the case of one-bit quantization, a variable is restricted to either one of two values—such as “0” and “1”. The one-bit quantization of block 414 may be aimed at discriminating between significant and insignificant correlation coefficient. By setting a predetermined threshold of between “−1” and “1”, correlation coefficients lower than the threshold may be filtered out, and be represented by the value “0”. The remaining correlation coefficients may be represented by the value “1”, indicating a cavitation event. In other words, it may be decided that a level of correlation which is lower than the predetermined threshold indicates a cavitation event.

In the experiment, the threshold was rigorously set to “0.9”—meaning that any correlation coefficient which was lower than “0.9” was deemed as indicating a cavitation event and was assigned a one-bit value of “1”. Conversely, any correlation coefficient which was higher than “0.9” was deemed as not indicating a cavitation event and was assigned a one-bit value of “0”. Persons of skill in the art will recognize that a threshold may be set to a different value, based on a preference of rigorousness; if it is desired to see only relatively high-intensity cavitation as a cavitation event, the threshold may be set closer to “−1”. If it is desired to see also relatively low-intensity cavitation as a cavitation event, the threshold may be set closer to “1”—as one essentially done in the experiment, for demonstration purposes only.

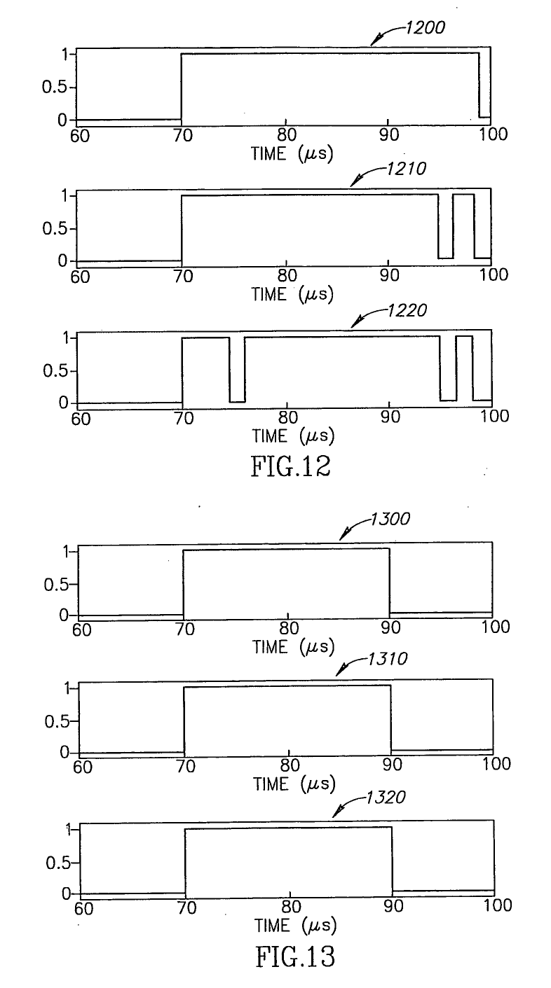

Reference is now made to FIG. 12, which shows three graphs 1200, 1210 and 1220. Graphs 1200-1220 are one-bit quantized versions of graphs 1000-1020 (that are computed with the small time window), respectively, of FIG. 10. Graphs 1200-1220 were created, in the experiment, using the aforementioned threshold value of “0.9”.

Graph 1200, for example, has a value of “0” between 60-70 μs, and a value of “1”—indicating a cavitation event—between approximately 70-99 μs. This corresponds to graph 1000 of FIG. 10, which shows a correlation coefficient of substantially “1” between approximately 60-70 μs, and various correlation coefficients lower than “1”, and in fact lower than “0.9”, between approximately 70-100 μs.

Similarly, graphs 1210-1220 show one-bit values corresponding to the correlation coefficients in graphs 1010-1020, respectively, of FIG. 10. Graph 1210, for example, shows two cavitation events—the first one between approximately 70-96 μs and the second one between approximately 97-99 μs. Graph 1220, as another example, shows three cavitation events—the first one between approximately 70-75 μs, the second one between approximately 76-97 μs, and the third one between approximately 98-99 μs.

Reference is now made to FIG. 13, which shows three graphs 1300, 1310 and 1320. Graphs 1300-1320 are one-bit quantized versions of graphs 1000-1020 (that are computed with the large time window), respectively, of FIG. 11. Graphs 1300-1320 were created, in the experiment, using the aforementioned threshold value of “0.9”. Since the correlation coefficients in graphs 1000-1020 of FIG. 11, between approximately 70-90 μs, are lower than “0.9”—the one-bit quantized versions in graphs 1300-1320 each show a cavitation event (“1”) over the entire range of 70-90 μs.

Referring now back to FIG. 4, a cavitation vector is optionally created in a block 416. The cavitation vector may be a statistical analysis of a plurality of one-bit quantized correlation coefficient graphs, such as graphs 1200-1220 of FIG. 12 and/or graphs 1300-1320 of FIG. 13. The statistical analysis may produce, for example, a statistically-significant indication of a location of cavitation, by averaging the plurality of one-bit quantized correlation coefficient graphs.

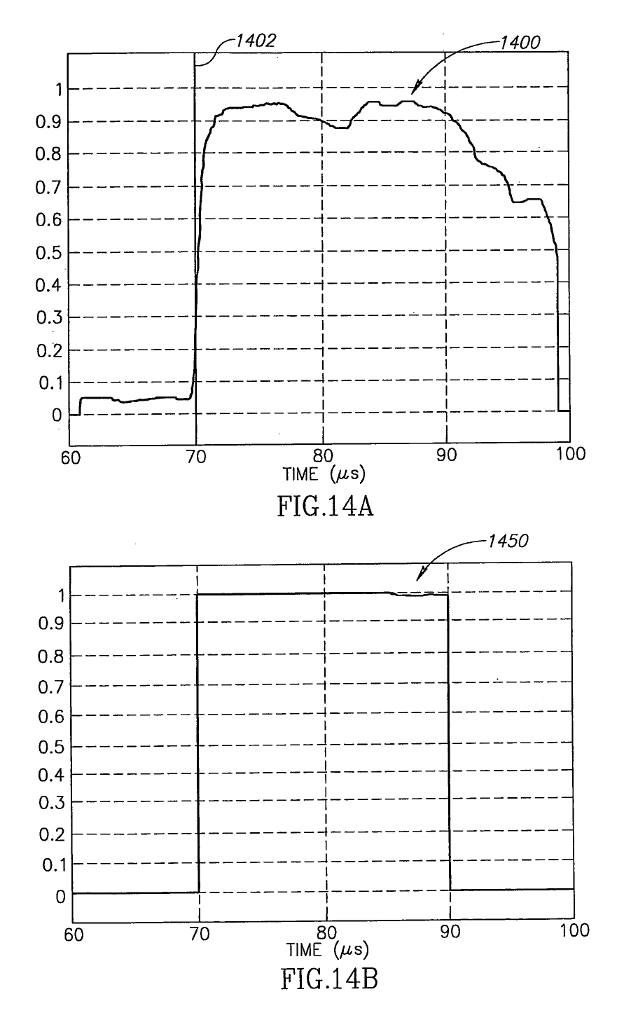

Reference is now made to FIG. 14A, which shows a cavitation vector 1400 created in the experiment by averaging 334 one-bit quantized correlation coefficient graphs. The correlation coefficients were computed with the small time window.

Therefore, cavitation vector 1400 may be used for determining a location of cavitation. A cavitation border 1402 marks a location of cavitation, which may, in fact, be a top border of a cavitation cloud containing micro-bubbles. The top border may be an edge of the cavitation cloud which is closest to the transducer. Cavitation border 1402 is marked based on an average of temporal beginnings of cavitation events in the 334 one-bit quantized correlation coefficient graphs. Cavitation border 1402 is positioned at approximately 70 μs, which translates to a distance of approximately 52.5 mm. This distance corresponds well to the focal length of the transducer used in the experiment, which was 54 mm.

A cavitation vector, such as cavitation vector 1400, may be created based on any number of one-bit quantized correlation coefficient graphs. The creation may be done in real time or in batch processing. Real time creation optionally involves computing a moving average of a pre-determined number of one-bit quantized correlation coefficient graphs. For example, a real time cavitation vector may show a vector of an N number of one-bit quantized correlation coefficient graphs computed based on previous acquisitions. With each additional one or more acquisitions made, the real time cavitation vector may change to reflect a cavitation border of the newly-acquired one or more acquisitions.

The batch processing method may include computing an average of a pre-determined number of one-bit quantized correlation coefficient graphs. For example, a batch-processed cavitation vector may show a vector of an M number of one-bit quantized correlation coefficient graphs computed based on previous acquisitions. The batch-processed cavitation vector is created only after the entirety of the M number of one-bit quantized correlation coefficient graphs are computed. An additional batch-processed cavitation vector may be created for each additional M number of one-bit quantized correlation coefficient graphs that are computed.

### Usage of Signal Analysis Results

The correlation coefficient(s), one-bit quantized correlation coefficient(s), cavitation vector(s), cavitation border(s) and/or any graph(s) thereof, are hereby referred to as “signal analysis result(s)”.

In an embodiment, one or more of the signal analysis result(s) are used for notifying a user, such as a histotripsy procedure caregiver, of one or more cavitation parameter(s). The cavitation parameter(s) are optionally one or more of the following:
Cavitation event indication: An essentially binary indication of existence and/or non-existence of cavitation at a particular moment in time or over a period of time. The cavitation event indication is optionally performed in real time, indicating existence or non-existence of cavitation substantially immediately following a cavitation event. Additionally or alternatively, the cavitation event indication is performed post factum, indicating existence or non-existence of cavitation substantially later than occurrence of the cavitation event(s). For example, the indication may be in the form of a treatment log, showing existence and/or non-existence of cavitation during a histotripsy session comprising a plurality of ultrasonic pulses. The cavitation event indication may be based on, for example, a computation of correlation coefficients such as the ones shown in FIGS. 7-8 and 10-11. Additionally or alternatively, indication may be based on, for example, one-bit quantized versions of correlation coefficients, such as the ones shown in FIGS. 12-13.
Cavitation intensity: A measure, accurate or estimated, of intensity of cavitation. As explained in the description of block 412 of FIG. 4, computation of correlation coefficients using, for example, a large time window, may enable drawing conclusions as to intensity of cavitation. A relatively low correlation coefficient may indicate a relatively high-intensity cavitation, whereas a relatively high level of cavitation may indicate a relatively low-intensity of cavitation.
Ratio of cavitation: A ratio between a number of radiated ultrasonic pulses and a number of cavitation events induced essentially by the pulses. The ratio of cavitation indication may be performed essentially in real time, substantially immediately following a radiated ultrasonic pulse and/or a cavitation event. Additionally or alternatively, the ratio of cavitation indication is performed post factum, after a portion of a histotripsy procedure or the entire procedure were completed.
Reference is now made to FIG. 14B, which shows an exemplary ratio of cavitation vector 1450 created in the experiment by dividing a number of radiated ultrasonic pulses by a number of cavitation events. Vector 1450 was created based on 334 one-bit quantized correlation coefficient graphs, some or all of which indicating cavitation events. As shown, the ratio of cavitation events is approximately “1” (which may also be referred to as “1:1”) between approximately 70 and 85 μs, and declines to approximately 0.98 (or “1:0.98”) between approximately 85 and 90 μs. This suggests that between approximately 70 and 85 μs, essentially each and every pulse, on average, induced cavitation, and between approximately 85 and 90 μs only about 98% of the pulses induced cavitation.
Location of cavitation: A one-dimensional (“1D”), two-dimensional (“2D”) and/or three-dimensional (“3D”) location value of one or more cavitation events.
1D location is optionally a distance of the one or more cavitation events from one or more histotripsy appliances, determined by measuring back-and-forth travel time of a radiated ultrasonic pulse. Since the medium speed of sound is known, and is approximately 1500 m/s, the distance may be easily computed. In case the back-and-forth travel of the radiated ultrasonic pulse is from a first source to a second, different, destination, such as when the pulse is radiated from a transducer and reflections of which are received in a cavitation detector—the distance between the transducer and the detector may be taken into consideration while computing the 1D location. Optionally, the 1D location is offset to show a depth of cavitation, indicating a depth of one or more cavitation events inside a tissue. For example, if the distance of a cavitation event from a histotripsy appliance is X mm, and the appliance is positioned Y mm above the patient's skin, the depth of cavitation may be expressed by X-Y mm.
Similarly, a 2D and/or a 3D location of one or more cavitation events may be computed. A 2D and/or a 3D location computation may be based on travel time readings of two or three cavitation detector(s)/transducer(s), respectively, located in different positions. If locations of the two or three detector(s)/transducer(s) is known, 2D or 3D location, respectively, of one or more cavitation events, may be computed.
The location of cavitation indication may be performed essentially in real time, substantially immediately following one or more cavitation events. Additionally or alternatively, the location of cavitation indication is performed post factum, after a portion of a histotripsy procedure or the entire procedure were completed.

The notification of the one or more cavitation parameter(s) may be visual, sonic, tactile and/or the like. Reference is now made to FIG. 15, which pictorially shows an exemplary treatment 1500 of a patient 1502 by a caregiver 1504. Caregiver 1504 may hold a transducer unit 1510 against an area of patient's 1502 body wherein destruction of adipose tissues is desired. For example, transducer unit 1510 may be held against the patient's 1502 abdomen 1508. Transducer unit 1510 may comprise one or more transducers (not shown) and/or one or more cavitation detectors (not shown). Transducer unit 1510 may be connected by at least one wire 1518 to a controller 1514.

Optionally, visual notification of one or more cavitation parameters is displayed on an exemplary monitor 1512 connected to controller 1514, and optionally functionally affixed to a pillar 1516. Those of skill in the art will recognize that a visual indication of one or more cavitation parameters may be displayed differently. For example, the visual indication may be performed by providing one or more light bulbs of any type (not shown), whose on/off and/or flashing status may indicate one or more cavitation parameters.

Optionally, sonic notification of one or more cavitation parameters is made by sounding one or more sounds through a speaker 1520 connected to controller 1514.

Optionally, tactile notification of one or more cavitation parameters is made by vibrating and/or otherwise exciting transducer unit 1510 held by caregiver 1504, so that the caregiver may feel the vibration/excitation and thus be notified of the one or more cavitation parameters.

In an embodiment, results of the signal analysis are used for automatically controlling one or more histotripsy procedure parameter(s). The histotripsy procedure parameter(s) are optionally one or more of the following:
Power: Electrical power and/or voltage used for radiating ultrasonic pulses. Generally, increasing power causes a stronger pulse, and vice versa. Therefore, if the signal analysis results indicate no cavitation and/or a relatively low-intensity cavitation, power may be increased to induce more cavitation and/or intensify the cavitation. Conversely, if the signal analysis results indicate many cavitation events and/or a relatively high-intensity cavitation, power may be decreased or even stopped to weaken the cavitation and/or to essentially cease it.
Number of pulses: Results of the signal analysis may be used for assessing, at a certain point in time during a treatment, if radiation of additional ultrasonic pulses is required for destroying an adipose tissue. For example, if the signal analysis results indicate no cavitation and/or a relatively low-intensity cavitation, more pulses may be radiated to induce more cavitation and/or intensify the cavitation. Conversely, if the signal analysis results indicate many cavitation events and/or a relatively high-intensity cavitation, radiation of pulses may be stopped to weaken the cavitation and/or to essentially cease it.
Focus: A focal point (or zone) of a transducer, where a majority of ultrasonic energy radiated by the transducer is essentially concentrated. Cavitation is likely to occur substantially at and/or around the focal point. Adjustment of the focal point may be done, for example, when using a multi-element transducer, such as multi-element transducer 300 (FIG. 3), and/or when using a time-reversal-based histotripsy system, such as systems 200 or 210 (FIG. 2A or 2B, respectively).
Reference is now made back to FIG. 15. Body contouring may be performed by emitting one or more ultrasonic pulses from transducer unit 1510 while it is held against a certain area of the patient's 1502 body. Then, transducer unit 1510 is optionally re-positioned above one or more additional areas and the emitting is repeated. Each position of transducer unit 1510 may be referred to as a “node”. A single body contouring treatment may include treating a plurality of nodes. Adjustment of the focal point may be advantageous, for example, while treating a certain node. When transducer unit 1510 is positioned at a certain node, focal point adjustment may enable destroying multiple areas of adipose tissues without essentially moving the transducer unit to a different node.
By way of example, if the signal analysis results indicate many cavitation events and/or a relatively high-intensity cavitation at a certain focal point, the focal point may be adjusted to perform treatment on a different zone of the adipose tissue.

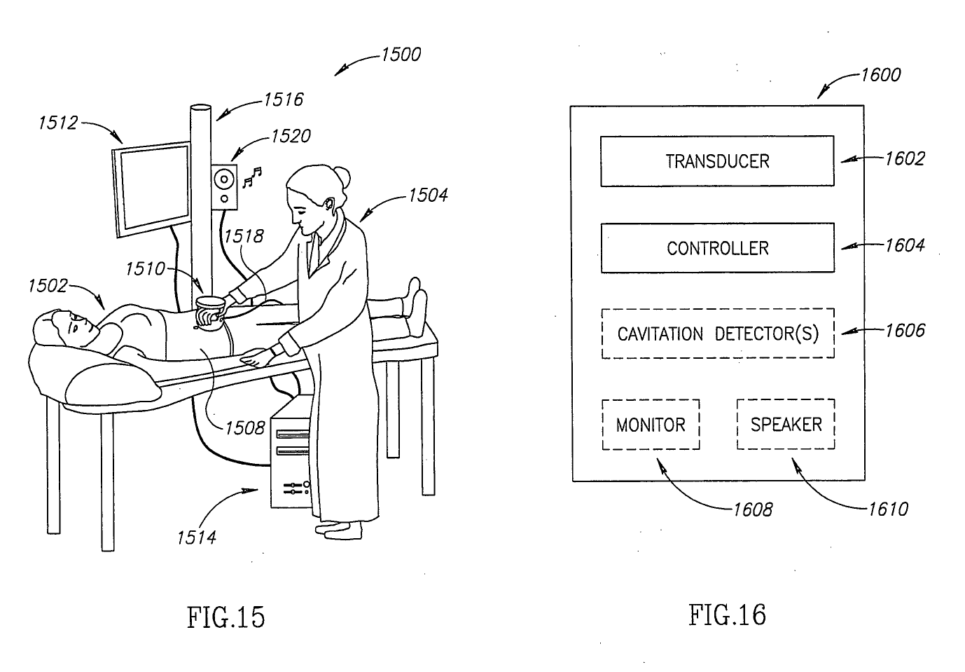

Reference is now made to FIG. 16, which shows a block diagram of an ultrasonic apparatus 1600 adapted to lyse an adipose tissue, in accordance with some embodiments. Ultrasonic apparatus 1600 includes a transducer 1602, a controller 1604, and optionally one or more cavitation detectors 1606. Transducer 1602 may be adapted to emit focused ultrasonic signals and/or to receive reflections of these signals. Controller 1604 may be adapted to process and/or analyze ultrasonic signals reflected substantially from an adipose tissue; optionally, the controller may be adapted to notify a user of one or more cavitation parameters; additionally or alternatively, the controller may be adapted to automatically control one or more histotripsy procedure parameters. Optional one or more cavitation detectors 1606 may be passive and/or active, as explained above.

Ultrasonic apparatus 1600 optionally further includes a monitor 1608, adapted to display notifications for a user and/or a speaker 1610 adapted to sound sonic notifications of the user.

In the description and claims of the application, each of the words “comprise” “include” and “have”, and forms thereof, are not necessarily limited to members in a list with which the words may be associated.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

What is claimed is:

1. A method for detecting cavitation in an adipose tissue, the method comprising computing a level of correlation between at least two received ultrasonic signals, wherein the level of correlation is indicative of cavitation.

2. The method according to claim 1, wherein a level of correlation lower than a predetermined threshold indicates a cavitation event.

3. The method according to claim 2, further comprising calculating a ratio between a number of cavitation events and a number of radiated ultrasonic signals, to determine a ratio of cavitation.

4. The method according to claim 2, further comprising computing an intensity of the cavitation event.

5. The method according to claim 2, further comprising computing a location of the cavitation event.

6. The method according to claim 5, wherein the location of the cavitation pertains to a one-dimensional location.

7. The method according to claim 5, wherein the location of the cavitation pertains to a two-dimensional location.

8. The method according to claim 5, wherein the location of the cavitation pertains to a three-dimensional location.

9. A method for automatically controlling a histotripsy appliance, the method comprising adjusting a histotripsy procedure parameter based on an indication of cavitation.

10. The method according to claim 9, wherein the histotripsy procedure parameter is an electrical power applied to the histotripsy appliance for radiating focused ultrasonic signals.

11. The method according to claim 9, wherein the histotripsy procedure parameter is a voltage applied to the histotripsy appliance for radiating focused ultrasonic signals.

12. The method according to claim 9, wherein the histotripsy procedure parameter is a focus of the histotripsy appliance.

13. The method according to claim 9, wherein the histotripsy procedure parameter is a number of focused ultrasonic signals radiated by the histotripsy appliance.

14. An ultrasonic apparatus for lysing an adipose tissue, the apparatus comprising:
a transducer adapted to radiate focused ultrasonic signals; and
a controller adapted to compute a level of correlation between at least two received ultrasonic signals, wherein the level of correlation is indicative of cavitation.

15. The ultrasonic apparatus according to claim 14, wherein a level of correlation lower than a predetermined threshold indicates a cavitation event.

16. The ultrasonic apparatus according to claim 15, wherein said controller is further adapted to compute a ratio between a number of cavitation events and a number of radiated ultrasonic pulses, to determine a ratio of cavitation.

17. The ultrasonic apparatus according to claim 15, wherein said controller is further adapted to compute an intensity of the cavitation event.

18. The ultrasonic apparatus according to claim 15, wherein said controller is further adapted to compute a location of the cavitation event.

19. The ultrasonic apparatus according to claim 18, wherein the location of the cavitation pertains to a one-dimensional location.

20. The ultrasonic apparatus according to claim 18, wherein the location of the cavitation pertains to a two-dimensional location.

21. The ultrasonic apparatus according to claim 18, wherein the location of the cavitation pertains to a three-dimensional location.

22. The ultrasonic apparatus according to claim 14, wherein said transducer is further adapted to function as a receiver for receiving said received ultrasonic signals.

23. The ultrasonic apparatus according to claim 22, further comprising a cavitation detector adapted to receive said received ultrasonic signals.

24. The ultrasonic apparatus according to claim 14, further comprising a cavitation detector adapted to receive said received ultrasonic signals

25. The ultrasonic apparatus according to claim 14, wherein said transducer is a multi-element transducer.

26. The ultrasonic apparatus according to claim 25, wherein at least one element of said multi-element transducer is adapted to receive the ultrasonic reflection signals.

27. The ultrasonic apparatus according to claim 14, wherein said transducer is associated with a time-reversal-based histotripsy system.

28. The ultrasonic apparatus according to claim 14, wherein said controller is further adapted to adjust a histotripsy procedure parameter.

29. The ultrasonic apparatus according to claim 28, wherein said histotripsy procedure parameter is an electrical power used for radiating the focused ultrasonic signals.

30. The ultrasonic apparatus according to claim 28, wherein said histotripsy procedure parameter is a voltage used for radiating the focused ultrasonic signals.

31. The ultrasonic apparatus according to claim 28, wherein said histotripsy procedure parameter is a focus of said transducer.

32. The ultrasonic apparatus according to claim 28, wherein said histotripsy procedure parameter is a number of the focused ultrasonic signals radiated by said transducer.

33. The ultrasonic apparatus according to claim 14, wherein said controller is further adapted to notify a user of a cavitation parameter.

34. The ultrasonic apparatus according to claim 33, wherein said cavitation parameter is a cavitation event.

35. The ultrasonic apparatus according to claim 33, wherein said cavitation parameter is an intensity of a cavitation event.

36. The ultrasonic apparatus according to claim 33, wherein said cavitation parameter is a ratio between a number of cavitation events and a number of radiated ultrasonic signals.

37. The ultrasonic apparatus according to claim 33, wherein said cavitation parameter is a location of a cavitation event.

38. The ultrasonic apparatus according to claim 33, further comprising a monitor adapted to display the notification of the cavitation parameter.

39. The ultrasonic apparatus according to claim 33, further comprising a speaker adapted to sound the notification of the cavitation parameter.

40. The ultrasonic apparatus according to claim 33, further comprising a vibrator adapted to sensorially relay the notification of the cavitation parameter to the user.
